SEQUENCE LISTING (7 sequences)

SEQ ID NO: 1 - protein, 235 aa, Artificial
  Amino acid sequence of the 6xHis-Ser237Ala-V848 mutant
MRGSHHHHHHGSVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNAGSPVFNEKNEVIGIHWGGVPNEFNGAVFINENVRNFLKQNIEDIHFANDDQKLN

SEQ ID NO: 2 - DNA, 708 bp, Artificial
  Coding nucleotide sequence including 6xHis linker and stop codon
atgagaggatcgcatcaccatcaccatcacggatccgttatattaccgaataacgatcgtcaccaaatcacagatacaacgaatggtcattatgcacccgtaacttatattcaagttgaagcacctactggtacatttattgcttccggtgtagttgtaggtaaagatactcttttaacaaataaacacgtcgtagatgctacgcacggtgatcctcatgctttaaaagcattcccttctgcaattaaccaagacaattatccaaatggtggtttcactgctgaacaaatcactaaatattcaggcgaaggtgatttagcaatagttaaattctcccctaatgagcaaaacaaacatattggtgaagtagttaaaccagcaacaatgagtaataatgctgaaacacaagttaaccaaaatattactgtaacaggatatcctggtgataaacctgtagcaacaatgtgggaaagtaaaggaaaaatcacttacctcaaaggcgaagctatgcaatatgatttaagtacaactggtggtaacgcaggttcacctgtatttaatgaaaaaaatgaagtgatcggaattcattggggcggtgtaccaaatgaatttaatggtgcggtatttattaatgaaaatgtacgcaacttcttaaaacaaaatattgaagatatccattttgccaacgatgaccaaaagcttaattaa

SEQ ID NO: 3 - DNA, 672 bp, Artificial
  Nucleotide sequence insert for cloning into vector pQE9
ggatccgttatattaccgaataacgatcgtcaccaaatcacagatacaacgaatggtcattatgcacccgtaacttatattcaagttgaagcacctactggtacatttattgcttccggtgtagttgtaggtaaagatactcttttaacaaataaacacgtcgtagatgctacgcacggtgatcctcatgctttaaaagcattcccttctgcaattaaccaagacaattatccaaatggtggtttcactgctgaacaaatcactaaatattcaggcgaaggtgatttagcaatagttaaattctcccctaatgagcaaaacaaacatattggtgaagtagttaaaccagcaacaatgagtaataatgctgaaacacaagttaaccaaaatattactgtaacaggatatcctggtgataaacctgtagcaacaatgtgggaaagtaaaggaaaaatcacttacctcaaaggcgaagctatgcaatatgatttaagtacaactggtggtaacgcaggttcacctgtatttaatgaaaaaaatgaagtgatcggaattcattggggcggtgtaccaaatgaatttaatggtgcggtatttattaatgaaaatgtacgcaacttcttaaaacaaaatattgaagatatccattttgccaacgatgaccaaaagctt

SEQ ID NO: 4 - DNA, 30 bp, Artificial
  Cloning primer PF1
cgcggatccgttatattaccaaataacgat

SEQ ID NO: 5 - DNA, 31 bp, Artificial
  Cloning primer PR1
cccaagcttttggtcatcgttggcaaaatgg

SEQ ID NO: 6 - DNA, 33 bp, Artificial
  Cloning primer PF2
agtacaactggtggtaacgcaggttcacctgta

SEQ ID NO: 7 - DNA, 33 bp, Artificial
  Cloning primer PR2
tacaggtgaacctgcgttaccaccagttgtact

(19) United States
(12) Patent Application Publication
(10) Pub. No.: US 2007/0298448 A1
(43) Pub. Date: Dec. 27, 2007

(54) Enrichment of Enzymatic Cleavage Products

(75) Inventors: Vukic Soskic, London (GB); Andre Schrattenholz, Mainz (DE)

Correspondence Address: SEED INTELLECTUAL PROPERTY LAW GROUP PLLC, 701 FIFTH AVE, SUITE 5400, SEATTLE, WA, 98104, US

(73) Assignee: ProteoSys AG, Mainz (DE)

(21) Appl. No.: 10/555,951

(22) PCT Filed: Apr. 27, 2004
(86) PCT No.: PCT/EP04/04404

§ 371 Date: Jan. 19, 2007

(30) Foreign Application Priority Data
May 7, 2003 (DE) 103 21 892.0
Jun. 4, 2003 (DE) 103 26 689.5

(51) Int. Cl. C12Q 1/37; C07H 21/04

(52) U.S. Cl. 435/23; 435/219; 536/23.2

(57) ABSTRACT

The invention relates to a method for the enrichment, isolation and/or identification of cleavage products of at least one enzyme from a sample. According to the invention, an enzymatically inactive mutant of a protease is used as an affinity material, said mutant furthermore maintaining its specific substrate nature. At least one cleavage product of the protease of which the mutant is used, and at least one cleavage product of the enzyme of which the cleavage products are to be analyzed, comprise at least one structural similarity.

The invention relates to a method for the enrichment, isolation and/or identification of enzymatic cleavage products, and to particular mutants and the use thereof.

The breakdown of proteins is an essential component of biological regulatory mechanisms like those taking place in all living organisms. Enzymes called proteases, which catalyze a cleavage, are crucially involved in the breakdown of proteins.

The enzymes which catalyze the hydrolytic cleavage (proteolysis) of the peptide linkage in proteins and peptides are called proteases. The proteases can be divided into those called proteinases (formerly: endopeptidases) and peptidases (formerly exopeptidases). The former cleave peptide linkages in the interior of a protein and thus produce peptides as cleavage products. The latter cleave proteins at the amino or carboxy end. Only proteases will ordinarily be mentioned hereinafter, with proteinases preferably being meant by this.

An important area of proteomic research is the identification of substrates of proteases and of the proteolytic products, i.e. the cleavage products, of these enzymes. This is an important precondition for making it possible to research the function of previously known and also novel proteases. Reference is made in this connection also to the “degradomics” field of research, which has the aim of identifying all the proteases of a proteome, and also the substrates which are cleaved by a particular protease. According to the definition, “degradomics” is the use of genomic and proteomic approaches for characterizing proteases and their substrates and inhibitors in a complex system as represented by a living organism.

Proteinases and their cleavage products in particular are of special interest for degradomics research. For example, research is particularly concentrated on the caspase family. The caspases play an important part in the controlled breakdown of various cellular substrates, and they are particularly involved in apoptotic processes, i.e. in breakdown processes associated with controlled cell death. The caspases are a highly conserved protease family having at least 12 human members. Because of their role in inflammatory processes and in the apoptosis of cells, the caspases are of enormous scientific interest. The caspases are included among the cysteine proteases, meaning that these proteases have cysteine, which is crucial for the proteolytic activity, at a critical site in the active center. Caspases are very specific proteases which cleave after an aspartic acid residue in their substrate. All cleavage products of caspases therefore have an aspartic acid residue at the C-terminal end of the peptide cleavage product (position P1). Glutamic acid is often present at position P3. Interesting conclusions can be drawn about the function and the role of the various caspases in the cell and in the organism by investigating the various cleavage products. It is possible inter alia by the general detection of cleavage products of the caspases to gain information about the activity of this multiple enzyme group without it being necessary to detect an individual representative of the caspases, which displays only very low activity in some circumstances.

The object of the invention is therefore to provide a method with which cleavage products of a particular enzyme or of a group of enzymes can be investigated with a small number of method steps. It is intended by investigating the cleavage products of an enzyme inter alia to be able to make statements about the activity of the enzyme(s) responsible for the cleavage.

This object is achieved by a method as set forth in claim 1. Claim 16 relates to a particular mutant of a protease and claim 25 to a corresponding nucleotide sequence. Claims 27 and 29 are concerned respectively with the use of the mutant and with a corresponding affinity matrix. Preferred embodiments are to be found in the dependent claims. The wording of all the claims is included in the description by reference.

It is possible by the process of the invention for cleavage products of at least one enzyme to be enriched, isolated and/or identified from a sample. This takes place with use of an enzymatically inactive mutant of a protease as affinity material, it being crucial for the method of the invention that the enzymatically inactive mutant of the protease continues to exhibit its substrate specificity. It is additionally important that the cleavage product(s) of the enzyme which are to be analyzed have at least a structural similarity to the hydrolytic cleavage products of the protease whose mutant is employed. The enzymatic inactivity is advantageous because, otherwise, cleavage products could be produced by the protease itself employed as affinity material, and would possibly falsify the results of the method of the invention.

In this method, firstly the sample containing the cleavage products to be detected is incubated with the enzymatically inactive mutant so that interactions between the cleavage products to be detected in the sample and the mutant can form. These interactions derive from the fact that the mutant has a high binding affinity for substrates having particular structural features. The cleavage products to be detected exhibit these structural features, so that they are specifically bound by this mutant. In a further step of the method, material which does not interact with the mutant can be removed. The cleavage products which have been bound by the mutant can then be analyzed. Whether separation of the interacting cleavage products from the mutant is worthwhile and possibly necessary before the actual analysis of the cleavage products depends on the specific design of the method and, in particular, on the analytical method.

The method of the invention is based on the fact that, on the one hand, the protease whose mutant is employed as affinity material has a high binding affinity for its own substrates and also for the products resulting from the proteolytic cleavage of the substrates. It is additionally necessary for this binding activity of the protease to be separable from its catalytic activity.

Such a separation of the catalytic activity from the binding activity is already known for the proteases trypsin and chymotrypsin. It is possible by a so-called anhydro modification in the catalytic center of these proteases to destroy the catalytic activity, i.e. the catalysis of hydrolytic cleavages, whereas the binding affinity for the cleavage products is retained. These forms, which are called respectively anhydrotrypsin and anhydrochymotrypsin, are therefore no longer able to cleave proteins. However, they can still bind their cleavage products. The cleavage products in the case of anhydrotrypsin are peptides having arginine and lysine at the C-terminal end. In the case of anhydrochymotrypsin they are peptides having hydrophobic amino acids at the C-terminal end. The anhydro mutants of trypsin and chymotrypsin can in this connection be achieved by a chemical modification or treatment where serine in the active center of the enzymes is replaced by alanine, i.e. the anhydro form of serine.

A similar separability of catalytic activity and binding affinity for substrates or cleavage products has been described for the protease ClpXP (Molecular Cell, Vol. 11, 671-683, 2003).

In a particularly preferred embodiment of the method of the invention, the enzyme whose cleavage products are to be enriched, isolated and/or identified is a protease, this protease preferably differing from the protease whose enzymatically inactive mutant is used as affinity material. This embodiment of the invention has the great advantage that the enzymatically inactive mutant of a protease can be employed as universal tool for investigating the cleavage products of any enzyme, as long as the cleavage products to be investigated have the appropriate structural features as are necessary for the binding activity of the employed enzymatically inactive mutant for particular substrates. Thus, a method which can be employed widely for proteomic research and which is based on the utilization of functional features is provided thereby. It is possible with the aid of the method of the invention to obtain inter alia results which allow conclusions to be drawn about the identity of different substrates or products of particular enzymes. In addition, a quantification of the activities of enzymes or whole enzyme families is made possible thereby.

In a preferred embodiment of the method of the invention, the structural similarities between the cleavage products to be investigated and the products which are bound by the protease whose enzymatically inactive mutant is employed comprises one or more coincident terminal amino acid residues, in particular C-terminal residues. For a large number of proteases whose mutants can be employed according to the invention, the binding affinity for their substrates derives from one or more particular C-terminal amino acids. For example, the V8 proteinase from Staphylococcus aureus (endoproteinase Glu(Asp)-C) shows a specific binding affinity for peptides which have glutamic acid (Glu) or aspartic acid (Asp) at the C-terminal end. The amino acid residues at other positions play a negligible part. An enzymatically inactive mutant of this V8 proteinase which still exhibits its substrate specificity is therefore suitable to be employed in the method of the invention for investigating cleavage products of other enzymes which have appropriate C-terminal amino acids or appropriate residues. This applies for example to the cleavage products of the caspases which, as mentioned at the outset, have aspartic acid at the C-terminal end. A particularly preferred embodiment of the invention is therefore one in which the structural similarity is a C-terminal glutamic acid and/or aspartic acid residue.

In a particularly preferred embodiment of the method of the invention, the enzymatically inactive mutant of the protease has an alteration in the active center. This destroys the catalytic activity according to the invention, although the binding activity is retained.

In a preferred embodiment of the method of the invention, the protease whose mutant is employed is a serine protease. Serine proteases are characterized in that they have serine at a critical site in the active center. Deletion or exchange of this serine destroys the enzymatic activity, whereas the substrate specificity is retained. These proteases are therefore particularly suitable according to the invention because it is possible by a single alteration which brings about an appropriate amino acid exchange to provide a mutant which can be employed according to the invention. These particularly suitable serine proteinases include for example the V8 proteinase already mentioned.

It is advantageous for the enzymatically inactive mutant to be an anhydro mutant. Particular preference is given in this connection to a serine to alanine exchange. Since in the serine proteases mentioned a serine in the catalytic center of the protease is responsible for the hydrolytic activity, the hydrolytic activity can be destroyed by such an anhydro mutation, while the substrate specificity is retained. It is, of course, also possible to employ other mutants of proteases according to the invention as long as they are enzymatically inactive, i.e. are no longer able to catalyze any hydrolytic cleavage, and still exhibit their substrate specificity.

In a particularly advantageous embodiment of the method of the invention, the enzymatically inactive mutant is employed in immobilized form. This substantially facilitates the carrying out of the method of the invention, since the incubation of the sample, the removal of material and, where appropriate, the separation of cleavage products can be carried out on a solid phase. It is particularly advantageous for the method to be carried out in the form of a column chromatography, in which case the enzymatically inactive mutant can be immobilized on a customary chromatography material such as, for example, Sepharose, agarose or Fraktogel. The immobilization can take place by customary methods. For example, the mutant can be coupled via a sequence of histidines to immobilized nickel ions (e.g. Ni-NTA agarose).

Analysis of the enriched cleavage products can take place by customary methods. A particularly preferred analysis is one using one- and/or two-dimensional polyacrylamide gel electrophoresis. The analysis can also be carried out using customary mass spectrometric methods. The mass spectrometry can also be combined with a polyacrylamide gel electrophoresis or other customary methods.

The actual method, i.e. the incubation of the sample and the removal of non-interacting material, can be carried out by carrying out a chromatography, in particular a column chromatography, for example a customary affinity chromatography. The analysis may additionally comprise one or more chromatography steps, especially column chromatography steps. It is additionally possible for example for a further fractionation of different enriched cleavage products to be achieved by one or more chromatography steps.

In a further embodiment of the invention, the cleavage products to be analyzed are modified during the method. This may entail in particular a further cleavage of the cleavage products, which is achieved for example by treatment with suitable enzymes. Tryptic digestion or the like is particularly suitable for this purpose. Such a modification takes place in particular with regard to an analysis of the cleavage products, with the cleavage products being fragmented further for example for a mass spectrometric analysis.

In a particularly preferred embodiment of the method of the invention, the protease whose enzymatically inactive mutant is employed is a V8 proteinase, for example a V8 proteinase from Staphylococcus aureus. A corresponding mutant, especially an anhydromutant of this enzyme, is particularly suitable according to the invention. It is to be used for an enrichment, isolation and/or identification of cleavage products having a glutamic acid or aspartic acid residue at the C terminus. It is particularly preferred in this connection for peptides having a C-terminal aspartic acid residue to be enriched or isolated and/or identified.

The method of the invention can advantageously be employed for investigating cleavage products of cysteine proteases. The method is very particularly suitable for investigating and characterizing cleavage products of one or more caspases. Caspases are very specific proteases whose cleavage products have aspartic acid at the C-terminal end. The use of an enzymatically inactive mutant of the V8 proteinase is thus particularly suitable for investigating cleavage products of caspases, because a corresponding mutant has a high affinity for cleavage products having a C-terminal aspartic acid residue.

The invention further encompasses an enzymatically inactive mutant of a protease, the substrate specificity being retained in the case of this mutant. Particular preference is given in this connection to a corresponding mutant of a serine protease, especially of a V8 proteinase, for example of a V8 proteinase from Staphylococcus aureus. Such a mutant can be employed with great advantage in the described method according to the invention. Reference is made to the above description concerning further features of this mutant.

In a particularly preferred embodiment of this mutant, it is the V8 proteinase mentioned, for example from Staphylococcus aureus, in which serine at position 237 is modified, exchanged or deleted. The serine at this position is preferably replaced by another amino acid, in particular by alanine. The serine to alanine exchange at position 237 preferably takes place in this case by a thymine to guanine base exchange at position 712. The serine at position 237 is the critical serine in the active center of the proteinase. An alteration at this site causes destruction of the catalytic, i.e. hydrolytic, activity, with retention of the substrate specificity.

The mutant of the invention can be prepared by chemical modification of the protease. However, it is particularly preferred for the mutant to be prepared by molecular biological methods.

The enzymatically inactive mutant of the invention may be characterized in that it has at least part of the amino acid sequence shown in SEQ ID No. 1. The invention further encompasses a corresponding mutant which is at least 70%, in particular at least 90% and preferably at least 99%, identical to the amino acid sequence shown in SEQ ID No. 1 or to one or more parts thereof. Included herein in particular are mutants which have the part or the parts of the amino acid sequence shown in SEQ ID No. 1 which are responsible for the substrate specificity. The invention also additionally encompasses those mutants which have such similarities with sequences of this type that they still bring about an appropriate substrate specificity in the sense according to the invention.

In a further preferred embodiment of this aspect of the invention, the enzymatically inactive mutant is further characterized in that it is present in immobilized form. Reference is made to the above description in this regard too.

The invention further encompasses a nucleotide sequence which codes for an enzymatically inactive mutant of a protease whose substrate specificity is retained. Reference is made to the above description concerning the further features of the protease encoded by this nucleotide sequence. The nucleotide sequence of the invention is characterized in particular in that it comprises at least part of the nucleotide sequence shown in SEQ ID No. 2. Particular preference is given in this connection to the parts of the nucleotide sequence which code for the region of the protease which are crucial for the substrate specificity.

The invention further encompasses the use of an enzymatically inactive mutant of a protease whose substrate specificity is retained as affinity material in a method for the enrichment, isolation and/or identification of cleavage products of at least one enzyme, where at least one cleavage product of the protease and at least one cleavage product of the enzyme have at least one structural similarity. Reference is made to the above description concerning further features of this use according to the invention.

Finally, the invention encompasses an affinity matrix for the enrichment, isolation and/or identification of enzymatic cleavage products.

This affinity matrix comprises an immobilized, enzymatically inactive mutant of a protease with retention of its substrate specificity. Reference is made to the above description in this regard too. This affinity matrix of the invention can be employed as universal tool for investigations in the proteomic sector and in degradomics research. It can be used for example to investigate changes in activity of whole enzyme families such as, for example, the caspases under various conditions.

The affinity matrix can for example be employed as simple column chromatography matrix comparable to a customary affinity chromatography. Loading of the sample to be investigated, and one or more washing steps can be followed by the cleavage products to be investigated being eluted, for example by changing the buffer conditions, and subsequently analyzed. The analysis can be carried out for example with a customary two-dimensional polyacrylamide gel electrophoresis. It is possible with such a method to obtain in a few steps results which provide information about enzymatic activities. It is additionally possible also to characterize and/or identify substrates and products of enzymes, especially of proteinases. The method of the invention provides an effective tool for enriching or purifying selectively specific classes of proteins or peptides which are of great interest in particular for proteomic research. Such a group-specific affinity tool opens up the possibility of drawing up profiles of proteins on the basis of their activity. It is possible thereby to investigate changes in the functional status of enzymes even when the quantitative level of the enzyme remains constant. It would further be possible with the aid of the invention to undertake investigations of the activity of inhibitors which influence various members of an enzyme family, for example the caspases. It is possible for this purpose to treat for example the intact cell or a cell extract with a potential inhibitor in order subsequently to analyze the activity of the enzyme family according to the invention in the manner described above.

Further features are evident from the examples in conjunction with the figures and the dependent claims. It is possible in this connection for the various features to be implemented each alone or in combination with one another.

The figures show:

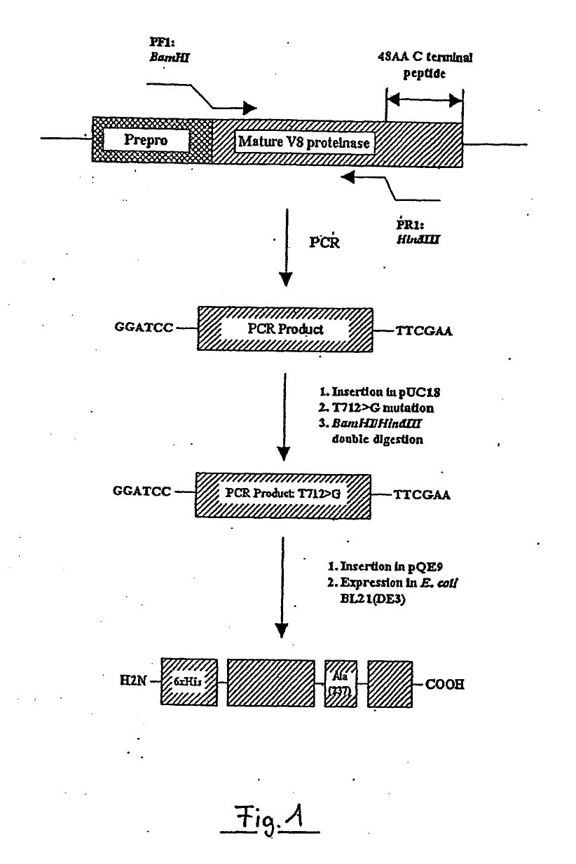

FIG. 1 Diagrammatic representation of the construction of Ser237Ala-V8Δ48.

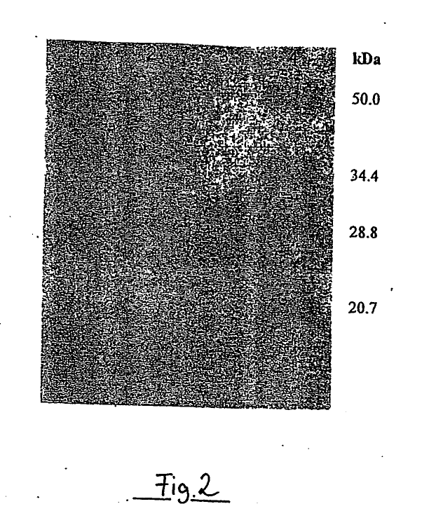

FIG. 2 Expression of the anhydro mutant of the V8 proteinase. The protein samples were fractionated on a 12% SDS polyacrylamide gel (SDS-PAGE) using a discontinuous buffer. The staining took place with Coomassie brilliant blue R-250. 1: complete cell lysate, 2: flow-through, 3: elution, 4: marker proteins.

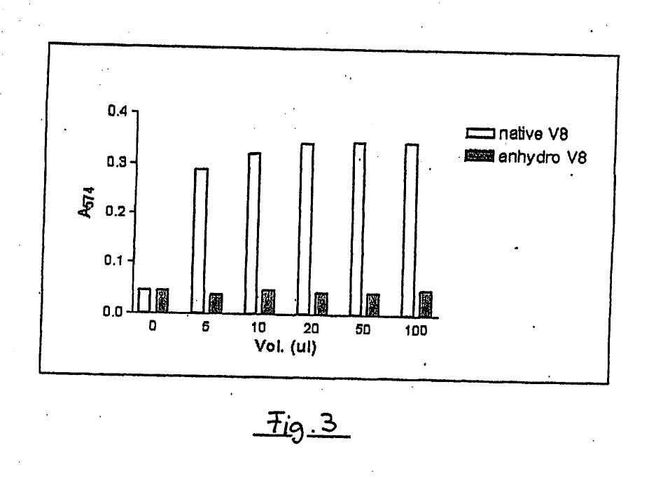

FIG. 3 Enzymatic activities of the wild type and the anhydro mutant of the V8 proteinase. The proteinase activity was measured at 30° C. in 0.1 M Tris-HCl pH 7.8, 0.01 M CaCl₂ with 0.4% universal protease substrate (Roche) in a volume of 0.2 ml. The activity was determined by measuring the absorption at 574 nm with a spectrophotometer for a time period of 10 min. The proteinase concentration was 1 μg/μl in each case.

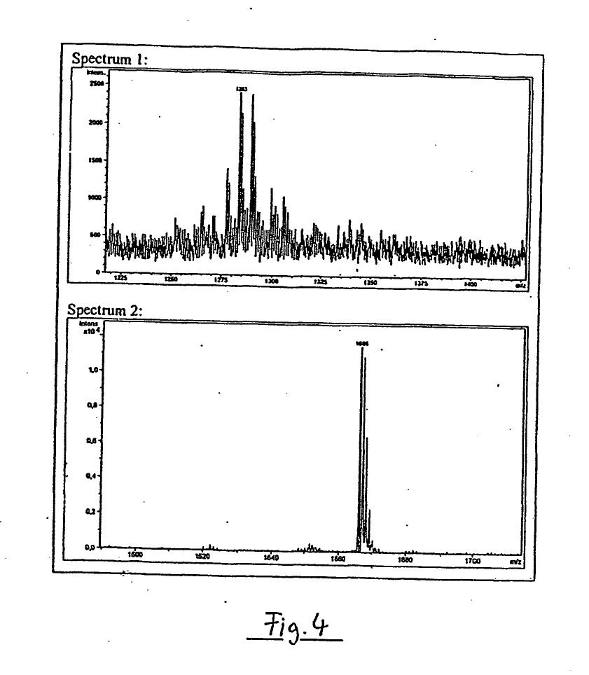

FIG. 4 Mass spectrometric measurement (MALDI-TOF) of the peptides eluted from the anhydro-V8-agarose. The Ni-NTA-agarose beads were loaded with Ser237Ala-V8Δ48 in 50 mM Tris-HCl, 300 mM NaCl, 1% CHAPS at pH 7.4. The neurotropic factor for retinal cholinergic neurones and [Cys(Bz)⁸⁴, Glu(OBz)⁸⁵]-CD₄(81-92) were incubated with the loaded agarose. The agarose was washed and eluted with 200 mM acetic acid. The samples were lyophilized in a Speed Vac and used for the MALDI analysis. Spectrum 1: eluate of the neurotropic factor for retinal cholinergic neurones, spectrum 2: eluate of Cys[(Bz)⁸⁴, Glu(OBz)⁸⁵]-CD₄(81-92).

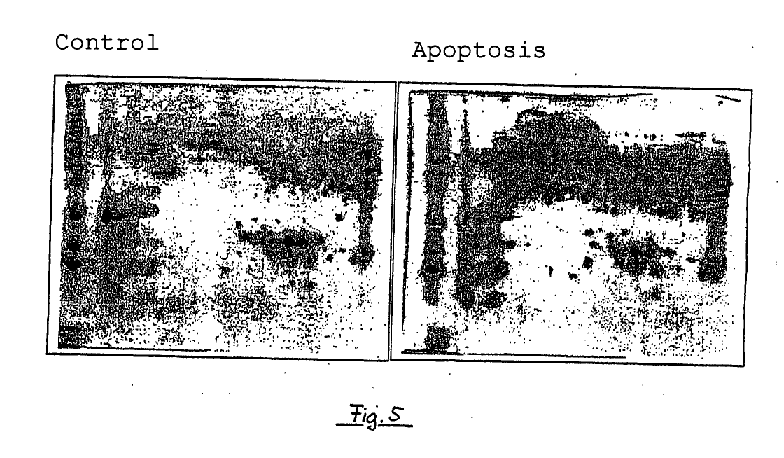

FIG. 5 Two-dimensional polyacrylamide gel electrophoresis of the eluate from the anhydro-V8-agarose. Readystrip IPG strips (pH 5-8) were loaded with 40 μg of cell extract. The isoelectric focusing (IEF) was carried out with up to 70 kVh. The SDS-PAGE was carried out with 11% polyacrylamide gels (70×70×1.0 mm) with constant current (40 mA) at 11° C. The second dimension was carried out until the bromophenol blue front reached the end of the gel. The gels were stained with silver nitrate. The left-hand gel shows the control, and the right-hand gel shows the fractionated cell extract of the cells with induced apoptosis.

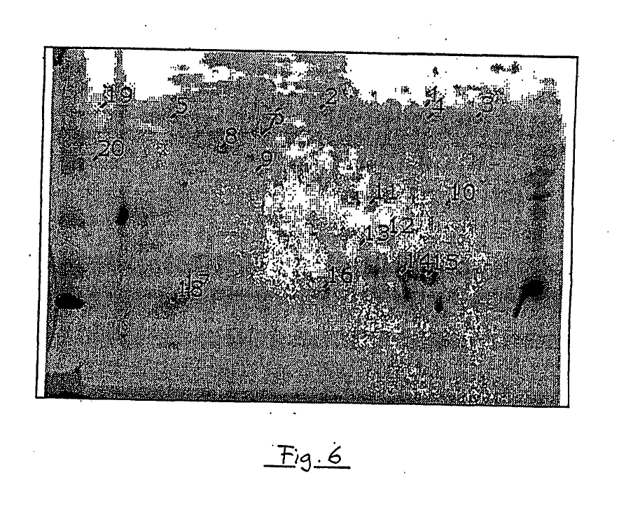

FIG. 6 Two-dimensional polyacrylamide gel of the fractionated cell extract of the cells with induced apoptosis, where the protein spots differing from the control gel (see FIG. 5) are marked with numbers.

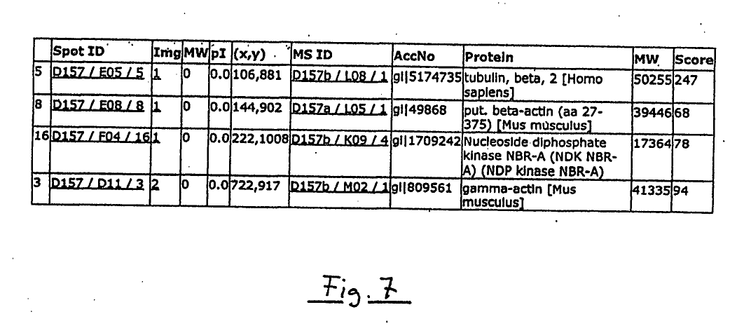

FIG. 7 Database comparison of mass spectrometric results of various spots from a two-dimensional polyacrylamide gel of cell extract from cells with induced apoptosis (see FIG. 6).

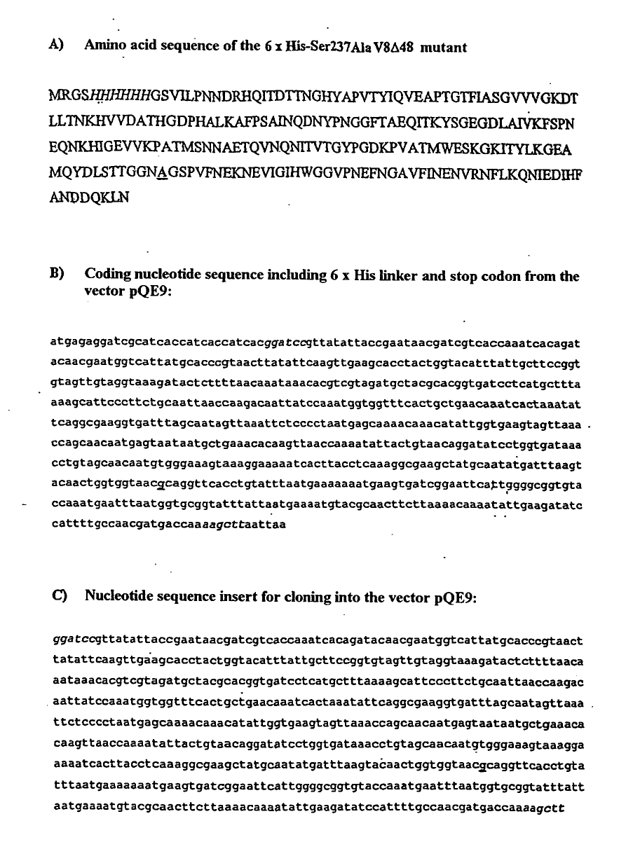

FIG. 8 Amino acid (A) and nucleotide sequences (B) and (C) of the 6xHis-Ser237Ala-V8Δ48 mutant. (A) shows the amino acid sequence of the mutant including the 6xHis linker (SEQ ID No. 1). (B) depicts the coding nucleotide sequence including the 6xHis linker and the stop codon from the vector pQE9 (SEQ ID No. 2). (C) lists the nucleotide sequence which was used as insert for cloning into the vector pQE9 (SEQ ID No. 3). In (A), 6xHis is shown in italics, and the alanine mutation is shown bold and underlined. In (B) and (C), the restriction cleavage sites (BamHI and HindIII) are shown in italics, and the point mutation (t to g) is shown bold and underlined.

### OVERVIEW OF THE SEQUENCE LISTING

| | |
|---|---|
| SEQ ID No. 1 | Amino acid sequence of the 6xHis-Ser237Ala-V8Δ48 |
| | mutant |
| SEQ ID No. 2 | Coding nucleotide sequence including 6xHis linker |
| | and stop codon |
| SEQ ID No. 3 | Nucleotide sequence insert for cloning into the vector |
| | pQE9 |
| SEQ ID No. 4 | Cloning primer PF1 |
| SEQ ID No. 5 | Cloning primer PR1 |
| SEQ ID No. 6 | Cloning primer PF2 |
| SEQ ID No. 7 | Cloning primer PR2 |

### EXAMPLES

1. Methods

1.1 Cloning of the V8Δ48 Protease

Staphylococcus aureus (ATCC 25923) was cultivated in LB medium at 37° C. overnight. Genomic DNA was purified with the RNA/DNA QIAGEN minikit starting from 1 ml of culture. The polymerase chain reaction (PCR) was carried out with the primers PF1 (SEQ ID No. 4) and PR1 (SEQ ID No. 5) (table 1), a dNTP mix and the PfuTurbo® DNA polymerase. Through these primers according to Yabuta et al. (Appl. Microbiol. Biotechnol. 44, 118-125 (1995)), a sequence which codes for amino acids 1 to 663 of the V8 protease (V8Δ48 protease) was amplified. The amplification products were fractionated by electrophoresis on a 1.2% agarose gel and stained with ethidium bromide. The resulting PCR products were purified, double-digested with BamHI and HindIII, and ligated into the vector pUC18 to result in the plasmid pUC18-V8Δ48. The ligation mixture was used to transfect Escherichia coli DH5α (competent cells). All the produced clones were checked by DNA sequencing.

TABLE 1

Sequences of the primers used for the cloning
and the mutagenesis of the anhydro-v8-proteinase.

| | | |
|---|---|---|
| | Restr. | |
| Primer sequence | Sites | Mutation |

Cloning primers

| | | | |
|---|---|---|---|
| PF1 | 5′-CGCGGATCCGTTATATTACCAAATAACGAT-3′ | BamHI | na |
| PR1 | 5′-CCCAAGCTTTTGGTCATCGTTGGCAAAATGG-3′ | HindIII | na |

Primers for site-directed mutagenesis

| | | | |
|---|---|---|---|
| PF2 | [symbol] | na | Ser [symbol] Ala |
| PR2 | 5′-TACAGGTGAACCTGCGTTACCACCAGTTGTACT-3′ | na | Ser [symbol] Ala |

1.2. Site-Directed Mutagenesis and Subcloning into the Vector pQE9

The site-directed mutagenesis of V8Δ48 was carried out using the QuikChange® XL site-directed mutagenesis kit and the plasmid pUC 18-V8Δ48. The mutation from serine (Ser) 237 to alanine (Ala) was achieved using the primers PF2 (SEQ ID No. 6) and PR2 (SEQ ID No. 7) (table 1). The vectors were isolated and sequenced. Positive clones were double-digested with BamHI and HindIII and ligated into the expression vector pQE9 to result in the plasmid pQE9-Ser237Ala-V8Δ48.

1.3. Expression and Purification of the V8 Protease from E. coli

The competent E. coli strain BL21(DE3) was transfected with the plasmid pQE9-Ser237Ala-V8Δ48. Freshly transfected cells with the plasmid were cultivated in 100 ml of LB (Luria Bertani) medium with 1 μg/ml ampicillin at 37° C. At a cell density of 0.6 (absorption at λ=660 nm), protein expression was induced by adding isopropyl thiogalactoside to a final concentration of 1 mM. After incubation for a further 3 h, the cells were harvested by centrifugation and suspended in 2 ml of lysis buffer (50 mM Tris-HCl, 300 mM NaCl, 1% CHAPS, pH 7.4). The cells were lysed by adding 0.1 ml of a solution containing lysozyme (20 mg/ml). The mixtures were incubated at 37° C. for 30 min and sonified for complete cell disruption. Incubation of the solutions was followed by centrifugation at 15000×g for 20 min. The supernatant was used further, and the pellet was discarded. A sample of 8 μl was taken from each supernatant for an SDS gel analysis.

Ni-NTA agarose beads were packed into 0.5 ml columns and loaded with the supernatant. The columns were washed with 5 column volume of lysis buffer and 5 column volume of lysis buffer with 20 mM imidazole. The bound protein was eluted with elution buffer (50 mM Tris-HCl, 300 mM NaCl, 1% CHAPS, 400 mM imidazole, pH 7.4). The protein content was determined by the BCA method with bovine serum albumin as calibration standard. The eluted fractions were analyzed by the Laemmli method on a 12% SDS-PAGE with discontinuous buffer. Staining took place with Coomassie brilliant blue R-250. The purified enzyme fractions were combined and desalted using NAP-5 gel filtration columns. The NAP-5 columns were equilibrated before use with 50 mM Tris-HCl, 1% CHAPS, 10% glycerol, pH 7.4. The samples were stored at −20° C. until used further.

1.4. Enzyme Activity Assay

The V8 protease activity was measured at 30° C. in 0.1 M Tris-HCl, pH 7.8, 0.01 M CaCl₂ with 0.4% universal protease substrate (Roche) in a volume of 0.2 ml. The activity of the enzyme was determined by observing the absorption at 574 nm over a period of 10 min.

1.5. Binding of N-Asp and N-Glu-peptides to the Immobilized Ser237Ala-V8Δ48 Protease

The Ni-NTA-agarose beads (20 μl) were loaded with 20 μg of Ser237Ala-V8Δ48 in 50 mM Tris-HCl, 300 mM NaCl, 1% CHAPS, pH 7.4. The agarose beads were washed with 2×200 μl of 0.1 M acetic acid and then 3×1 ml of 50 mM Na phosphate, 300 mM NaCl, 1% CHAPS, pH 7.2. Neurotropic factor for retinal cholinergic neurons and [Cys(Bz)⁸⁴, Glu(OBz)⁸⁵]-CD₄(81-92), in each case 20 μl of a 1 mg/ml solution of each peptide in the same buffer, were incubated with the loaded agarose beads, shaking constantly at room temperature for 20 min. [Cys(Bz)⁸⁴, Glu(OBz)⁸⁵]-CD₄(81-92) is the short amino acid sequence AA81-92 of the CD4 protein which is derivatized at position 84 (Cys) with Bz and at position 85 (Glu) with OBz. The beads were washed three times with 500 μl of ice-cold buffer each time and 500 μl of 20 mM NH₄HCO₃, pH 8.4 each time, and eluted with 20 μl of 200 mM acetic acid. The samples were lyophilized in a Speed Vac and used for the mass spectrometric analysis (MALDI-TOF).

1.6. Cell Culture

The NRK-49F rat kidney fibroplast cell line was cultivated in Dulbecco's modified Eagle's medium (Ham's F12 nutrient mix with 10% fetal calf serum (FCS), 1% antibiotics-antimycotics (100×solution with 10000 U/ml penicillin G, 10 mg/ml streptomycin and 25 μl/ml amphotericin B) in a humidified atmosphere with 5% CO₂ at 37° C. The cells were cultivated in 10 cm culture dishes to subconfluence and split in the ratio 1:5 using trypsin-EDTA. For all experiments, 80% confluent NRK-49F cells were rested by incubating in corresponding medium with 0.5% FCS for 24-48 h. The cells were stored by freezing in 90% FCS, 10% DMSO in liquid nitrogen.

1.7. Induction of Apoptosis and Preparation of the Cell Extract

Apoptosis was induced in 80% confluent cells by adding 1 M hydrogen peroxide to a final concentration of 1 mM. Control cells were incubated without hydrogen peroxide for a corresponding period. The cells were lysed in hypotonic buffer (10 mM Na phosphate, pH 7.2, 1% Triton X-100, 1× complete protease inhibitors) and the insoluble cell detritus was removed by centrifugation at 10000×g for 20 min. 5 M NaCl was added to the supernatant to achieve a final concentration of 300 mM. The samples obtained in this way were loaded onto 100 μl packed Ni-NTA-agarose columns which were loaded with Ser237Ala-V8Δ48 protease as described above. The beads were washed 3× with 2 ml of ice-cold buffer with 300 mM NaCl, and eluted with 100 μl of 1% SDS in 10 mM Tris-HCl, pH 7.4, 300 mM NaCl at 80° C. for 5 min. The salts were removed using millipore BIOMAX 5K ultrafiltration membrane devices, and the samples were diluted in electrophoresis sample buffer.

1.8. Two-Dimensional Gel Electrophoresis

Ready-to-use Readystrip IPG strips (pH 5-8) from Bio-Rad were rehydrogenated with 50 μl of cell extract overnight. The isoelectric focusing was carried out up to a total of 70 kVh. Before the SDS gel electrophoresis, the IPG strips were incubated in a solution of 20 mg/ml of dithiothreitol (DTT) in equilibration buffer for 20 min and then put into a solution of 45 mg/ml iodoacetamide in the same buffer for 20 min. SDS-PAGE was carried out using an 11% polyacrylamide gel (70×70×1.0 mm) at a constant current of 40 mA at 11° C. The second dimension was carried out until the bromophenol blue front had reached the end of the gel. The gels were stained with silver by the method of Shevchenko et al. (Anal. Chem. 68, 850-858 (1996)).

1.9. Sample Preparation for the MALDI-TOF Mass Spectrometry

In order to obtain mass spectrometric peptide maps of the proteins, 0.5 μl aliquots of the generated cleavage products were distributed on the sample carrier, and 0.5 μl of a solution of α-cyano-4-hydroxy-cinnamic acid in 35% acetonitrile/0.1% trifluoroacetic acid was added.

1.10. MALDI-TOF Mass Spectrometry

The samples were analyzed in an Autoflex MALDI-TOF mass spectrometer (Bruker, Germany). All the spectra were recorded in a positive ion reflector mode. Typically, the first 10 shots of a new spot were discarded, and the next 200 shots were recorded.

1.11. Calibration of the MALDI-TOF Spectra

The external standards used for calibration were human angiotensin I and II, adrenocorticotropic hormone, [Glu]-fibrinopeptide B, rennin substrate tetradecapeptide and the insulin B chain. The amount of each peptide was 0.25 pmol per spot.

2. Results

The coding regions of the V8Δ48 protease were amplified by a polymerase chain reaction using the genomic DNA from S. aureus by the method of Yabuta et al. The resulting product was ligated via the BamHI/HindIII restriction sites into the plasmid pUC18. It was shown by double-stranded sequencing that the coding region is identical to the sequence described in earlier studies.

A site-directed mutagenesis was employed to produce a Ser-237 to Ala mutation (T712>G) in the pUC18 plasmid having the V8 protease gene. This step was checked by DNA sequencing. The identified colony which harbored this mutation was cultivated in LB medium, and the corresponding plasmid was isolated and digested with BamHI and HindIII. The resulting Ser237Ala-V8Δ48 gene was subcloned into the expression vector pQE9 by use of the BamHI/HindIII restriction sites and used to transfect E. coli BL21 (DE3).

The mutant Ser237Ala-V8Δ48 with an N-terminal histidine tag (FIG. 1) was successfully expressed in E. coli BL21 (DE3) on use of isopropyl β-D-thiogalactopyranoside at 37° C. for 3 h. Ni-NTA agarose columns were employed to purify the mutant. The protein fractions containing Ser237Ala-V8Δ48 were desalted as soon as possible after the purification by NAP5 columns which were equilibrated with 25 mM Tris-HCl, pH 7.4, 1% Triton X-100 and 100 mM NaCl. The purity of the enzyme was investigated by SDS-PAGE, with a single band of 26 kDA with a purity of more than 95% being observed (FIG. 2). Almost the whole amount of the V8Δ48 proteinase mutant was expressed as soluble protein, which could be purified in a single step by use of Ni-NTA agarose affinity columns.

The expressed purified protein showed no proteolytic activity. The wild type of the V8 proteinase was employed as positive control in the same mixture (FIG. 3).

The neurotropic factor for retinal cholinergic neurons and [Cys(Bz)⁸⁴, Glu(OBz)⁸⁵]-CD₄(81-92) were used to test the binding properties of Ser237Ala-V8Δ48 for peptides having aspartic acid or glutamic acid at the C-terminal end. The mutant immobilized on Ni-NTA-agarose beads was incubated with each of the peptides and, after washing the beads, the bound peptides were eluted with 200 mM acetic acid. Both peptides were detected in the eluates by MALDI-TOF (FIG. 4). Calibration peptides were employed for the MALDI-TOF mass spectrometry as a check of this experiment. No signals were detected in the MALDI-TOF spectrum with these control peptides. In order to test the binding properties of this novel affinity material for cleavage products of caspases, once again purified Ser237Ala-V8Δ48 immobilized on Ni-NTA-agarose was used. Apoptosis was induced in NRK-49F cells by using hydrogen peroxide for this experiment. After isolation, the proteins and peptides were put onto the Ni-NTA-agarose with Ser237Ala-V8Δ48. After washing the unbound material, the bound peptides were eluted with an SDS solution at an elevated temperature. Non-apoptotic NRK-49F cells were used as a check of this experiment. Both samples were analyzed by 2D PAGE, with marked differences being observable in the protein and peptide patterns (FIG. 5). Many of the protein spots in FIG. 5 were observable both in the control and in the gel of the apoptotic cell extract. The reason for this is that all the proteins having aspartic acid or glutamic acid at the C-terminal end were bound by the affinity material. All these proteins can be regarded as background. The protein spots observable only in the gel of the apoptotic cell extract are the cleavage products attributable to a caspase activity.

To identify the cleavage products attributable to a caspase activity, corresponding spots on the two-dimensional gel were marked (FIG. 6) and cut out of the gel. The proteins or peptides were obtained from the gel by tryptic digestion. The resulting fragments were subjected to a MALDI-TOF mass spectrometry (Vogt et al., 2003. Rapid communications in mass spectrometry 17: 1273-1282). The masses found were compared with data from a database (FIG. 7) and, in this way, the proteins from the two-dimensional gel were identified. This revealed that the protein labeled as spot 5 in FIG. 6 is β-tubulin, the protein labeled as spot 8 is β-actin, and the protein labeled as spot 16 is nucleoside-diphosphate kinase (nm23). These proteins are described as caspase substrates in the literature (e.g. J. Urol. May 2003; 169 (5): 1729-1734; J. Neuroscience Mar. 1, 2003; 23 (5): 1742-1749; J. Neuroscience Research Oct. 15, 2002; 70 (2): 180-189; J. Comp. Neurol. Oct. 7, 2002; 452 (1): 65-79; J. Comp. Neurol. Aug. 28, 2000; 424 (3): 476-488; Brain Res. Mol. Brain Res. Jan. 10, 2000; 75 (1): 143-149; Cell Mar. 7, 2003; 112 (5): 659-672; Cell Mar. 7, 2003; 112 (5): 589-591; Blood Apr. 15, 2003; 101 (8): 3212-3219). The protein labeled as spot 3 was identified as γ-actin (Eur. J. Biochem. January 2003; 270 (2): 342-349; Arch. Dermatol. Res. June 2001; 293 (6): 283-290; Mol. Endocrinol. October 1991; 5 (10): 1381-1388). This protein shows great homology with β-actin, so that it can be assumed that γ-actin is likewise a caspase substrate.

1. A method for the enrichment, isolation and/or identification of cleavage products of at least one enzyme, from a sample using an enzymatically inactive mutant of a V8 proteinase with retention of the substrate specificity as affinity material, where at least one cleavage product of the protease and at least one cleavage product of the enzyme have at least one structural similarity, comprising:
incubation of the sample with the enzymatically inactive mutant to form interactions between possible cleavage products of the enzyme in the sample and the mutant,
removal of non-interacting material,
where appropriate, separation of the interacting cleavage products from the mutant,
where appropriate, analysis of the cleavage products.

2. The method as claimed in claim 1, wherein the enzyme is a protease different from the protease whose enzymatically inactive mutant is used.

3. The method as claimed in claim 1, wherein the at least one cleavage product of the V8 proteinase has at least one identical terminal amino acid as the at least one cleavage product of the enzyme.

4. The method as claimed in claim 3, wherein the at least one identical terminal amino acid is C-terminal amino acid, glutamic acid or aspartic acid.

5. The method as claimed in claim 1, wherein the enzymatically inactive mutant of the V8 proteinase has an alteration in the active center.

6. The method as claimed in claim 1, wherein the V8 proteinase is a serine protease.

7. The method as claimed in claim 1, wherein the enzymatically inactive mutant is an anhydro mutant.

8. The method as claimed in claim 1, wherein the mutant has a replacement of serine by alanine.

9. The method as claimed in claim 1, wherein the enzymatically inactive mutant is immobilized.

10. The method as claimed in claim 1, wherein the analysis is carried out using polyacrylamide gel electrophoresis or mass spectrometry.

11. The method as claimed in claim 1, wherein the analysis comprises at least one chromatography step.

12. The method as claimed in claim 1, wherein the cleavage products are modified during the method.

13. An enzymatically inactive mutant of a V8 proteinase, wherein the substrate specificity of the V8 proteinase is retained.

14. The mutant as claimed in claim 13, wherein it has an alteration in the active center.

15. The mutant as claimed in claim 13, wherein it is an anhydro mutant.

16. The mutant as claimed in claim 13, wherein it has a replacement of serine by alanine.

17. The mutant as claimed in claim 13, wherein the serine at position 237 is replaced.

18. The mutant as claimed in claim 13, wherein it has at least part of the amino acid sequence shown in SEQ ID No. 1.

19. The mutant as claimed in claim 13, wherein it has an amino acid sequence which is at least 70% identical to at least part of the amino acid sequence shown in SEQ ID No. 1.

20. The mutant as claimed in claim 13, wherein it is immobilized.

21. A nucleotide sequence which codes for an enzymatically inactive mutant of a V8 proteinase as claimed in claim 13.

22. The nucleotide sequence as claimed in claim 21, wherein it has at least part of the nucleotide sequence shown in SEQ ID No. 2.

23. (canceled)

24. (canceled)

25. An affinity matrix for the enrichment, isolation and/or identification of enzymatic cleavage products, comprising an immobilized, enzymatically inactive mutant of a V8 proteinase with retention of the substrate specificity.
